(19) United States
(12) Patent Application Publication
(10) Pub. No.: US 2025/0082821 A1
(43) Pub. Date: Mar. 13, 2025

(54) COMPOSITIONS AND RELATED METHODS

(71) Applicant: Boston Scientific Scimed, Inc., Maple Grove, MN (US)

(72) Inventors: Lauren Sfakis LYDECKER, Millbury, MA (US); Megan CHROBAK, Westford, MA (US); Jessica Lynne GRIMSBY, Watertown, MA (US)

(73) Assignee: Boston Scientific Scimed, Inc., Maple Grove, MN (US)

(21) Appl. No.: 18/828,178

(22) Filed: Sep. 9, 2024

Related U.S. Application Data
(60) Provisional application No. 63/582,335, filed on Sep. 13, 2023.

(51) Int. Cl. A61L 27/20; A61L 27/44; A61L 27/46; A61L 27/52

(52) U.S. Cl. CPC A61L 27/20; A61L 27/446; A61L 27/46; A61L 27/52; A61L 2400/06

(57) ABSTRACT

Compositions and methods of preparation and use thereof are described. The composition may include chitosan crosslinked with tripolyphosphate and calcium, optionally wherein the composition is prepared by combining particles that include chitosan and tripolyphosphate with calcium ions, such as calcium ions from a calcium salt.

### CROSS-REFERENCE TO RELATED APPLICATIONS

This application claims the benefit of priority to U.S. Provisional Application No. 63/582,335, filed on Sep. 13, 2023, which is incorporated by reference herein in its entirety.

### TECHNICAL FIELD

The present disclosure relates generally to compositions useful for separating tissues and/or as filler materials and related methods of preparation of use. For example, the present disclosure includes crosslinked chitosan compositions useful as a spacer or filler between tissues.

### BACKGROUND

Filler or lifter materials may be utilized for various medical conditions and procedures. For example, patients undergoing urologic procedures may benefit from separation of tissue layers, e.g., between a prostate and rectum during radiation therapy for prostate cancer to protect the rectum from radiation exposure. Patients diagnosed with endoscopic conditions and/or undergoing endoscopic procedures may also have need for materials for filling fistulas and/or separation of tissue. A fistula is an abnormal connection between organs, vessels, or other tissues. Fistulas may be filled in an effort to repair them. Biocompatible materials may also be used as a lifting agent, e.g., to separate tissues during removal of polyps, adenomas, gastrointestinal lesions, or early-stage cancerous tissue.

### SUMMARY

The present disclosure includes composition and methods of preparation and use thereof. For example, the present disclosure includes a composition comprising chitosan crosslinked with tripolyphosphate and calcium, optionally wherein the composition is prepared by combining particles comprising chitosan and tripolyphosphate with calcium ions, such as calcium ions from a calcium salt. The calcium salt may comprise calcium chloride, for example. The composition may be prepared by combining a suspension of the particles comprising chitosan and tripolyphosphate with the calcium salt, wherein the suspension is aqueous. According to some aspects of the present disclosure, the suspension further comprises polyethylene glycol, a surfactant, or both. In some examples, the composition is prepared by combining the particles comprising chitosan and tripolyphosphate with an aqueous solution of calcium chloride, optionally wherein the aqueous solution of calcium chloride is supersaturated. The composition may be in the form of a gel, e.g., a crosslinked matrix of gel particles. Crosslinking of the calcium and the particles may be reversible, such that adding saline solution breaks up the crosslinked structure of the composition. In some aspects, the particles comprising chitosan and tripolyphosphate have an average particle diameter ranging from about 100 μm to about 700 μm, such as from about 100 μm to about 300 μm. In some examples, the composition comprises about 10% to about 99% by weight of the chitosan crosslinked with tripolyphosphate and calcium, with respect to the total weight of the composition. The present disclosure also includes use of the composition(s) described above and elsewhere herein, e.g., as a lifting agent, spacer material, or filler material such as an embolic filler or fistula filler. For example, the composition may be administered to a target site of a subject by combining the particles comprising chitosan and tripolyphosphate with the calcium ions at the target site to form the composition. The target site may comprise soft tissue, for example, wherein the composition separates tissue layers. In some examples, the target site comprises a fistula and the composition at least partially or completely fills the fistula. The composition may be administered to the target site using a catheter. Optionally, the particles comprising chitosan and tripolyphosphate and the calcium ions may be administered to the target site at the same time.

The present disclosure also includes a method of treating a subject, the method comprising administering a composition to a target site of the subject, wherein administering the composition includes combining particles that comprise chitosan and tripolyphosphate with calcium ions to form a crosslinked material. The calcium ions may be provided by a calcium salt solution, such as, for example, calcium ions of a calcium chloride salt solution. According to some aspects, the particles comprising chitosan and tripolyphosphate have an average particle diameter ranging from about 100 μm to about 700 μm. The composition may be administered via a catheter. According to some aspects of the present disclosure, administering the composition includes combining the particles with the calcium ions at the target site at the same time. The method may further comprise removing at least a portion of the composition from the target site by adding saline solution to the composition. In at least one exemplary method, the target site is a fistula, and the composition at least partially or completely fills the fistula. In at least one exemplary method, the target site is soft tissue, and the composition separates tissue layers. The target site may be between a pancreas and an abdominal cavity or gallbladder, for example, of the subject.

The present disclosure also includes a method of preparing a composition, the method comprising combining particles that comprise chitosan and tripolyphosphate with a calcium salt solution to form the composition, wherein the composition comprises chitosan crosslinked with tripolyphosphate and calcium. The composition may be prepared by combining an aqueous suspension of the particles with the calcium salt solution. Optionally, the aqueous suspension further comprises polyethylene glycol, a surfactant, or both. In some examples, the calcium salt solution comprises calcium chloride and/or the calcium salt solution is supersaturated. In some examples, the particles comprising chitosan and tripolyphosphate have an average particle diameter ranging from about 100 μm to about 700 μm.

The present disclosure also includes a method of treating a subject, the method comprising administering a composition to a target site of the subject by combining an aqueous suspension comprising particles that comprise chitosan and tripolyphosphate with a calcium salt solution at the target site to form a crosslinked material. In some examples, the aqueous suspension further comprises polyethylene glycol, a surfactant, or both. According to some aspects, the target site is a fistula or soft tissue of the urinary tract or gastrointestinal tract. For example, the composition may be administered via a catheter.

### BRIEF DESCRIPTION OF THE FIGURES

The accompanying drawings, which are incorporated in and constitute a part of this application, illustrate various exemplary embodiments and together with the description, serve to explain the principles of the disclosed embodiments.

FIG. 1 shows an exemplary reaction schematic, according to some aspects of the present disclosure.

FIG. 2 shows chitosan particles (left) and an exemplary composition (right) prepared from the chitosan particles, as discussed in Example 1.

FIGS. 3A-3B show micrographs of an exemplary composition prepared as discussed in Example 1.

### DETAILED DESCRIPTION

Aspects of the present disclosure are described in greater detail below. The terms and definitions provided herein control, if in conflict with terms and/or definitions incorporated by reference.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass+5% of a specified amount or value.

Embodiments of the present disclosure include compositions comprising biocompatible materials and related methods of preparing and administering the compositions to a target site. The compositions herein may be utilized for various medical conditions and procedures, e.g., as a lifting agent, spacer material, or filler material. For example, patients undergoing urologic or endoscopic procedures may benefit from increased spacing between various organs, e.g., between the prostate and rectum during radiation therapy for prostate cancer to protect the rectum from radiation exposure. Further, for example, fistulas may be at least partially or completely filled in an effort to repair them. The compositions herein also may be used as a lifting agent, e.g., to separate tissue layers, which may aid in removing polyps, adenomas, gastrointestinal lesions, and/or early-stage cancerous tissue. The compositions herein also may be useful as an embolic filler, e.g., for blood vessels in a variety of hypervascularized tumors, including uterine fibroids and arteriovenous malformations. The compositions herein may be formulated as a crosslinked material, e.g., a gel, with the desired density and/or viscosity to maintain structural integrity of the material at the target site. As discussed further below, the crosslinking may be reversible, such that the structure of the crosslinked matrix or gel may be broken down, e.g., dissolved or otherwise disrupted. This characteristic may be useful in situations in which the composition is not administered to the proper site and it is desired for the misplaced composition to be removed.

The compositions herein may comprise crosslinked chitosan. For example, the composition may comprise chitosan crosslinked with tripolyphosphate (TPP) and calcium (e.g., calcium ions Ca²⁺).

Chitosan is a linear polysaccharide formed of glucosamine units derived from chitin, the structural component of crustacean exoskeletons:

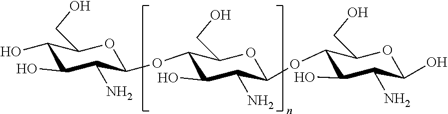

Chitosan is typically prepared by deacetylation of chitin with an alkaline reagent such as sodium hydroxide, yielding a water-soluble material. Chitosan may be crosslinked with TPP to form particles. An exemplary process for preparing such particles is described in US 2021/0322629 A1.

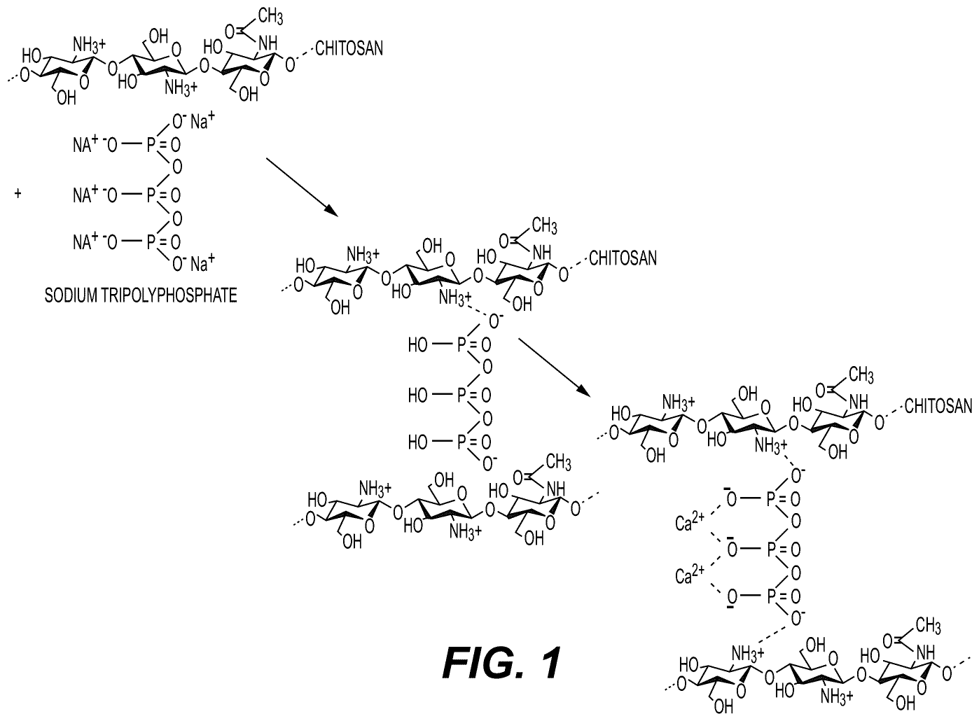

To prepare the compositions herein, chitosan particles (e.g., particles comprising chitosan crosslinked with TPP) may be combined with Ca²⁺ ions capable of bonding with available phosphate groups of the particles (e.g., phosphate groups of the TPP) to form a crosslinked matrix. This is illustrated in the final step of the exemplary reaction schematic shown in FIG. 1. The source of calcium ions may be a biocompatible salt, such as calcium chloride.

According to some aspects of the present disclosure, the composition may be prepared by combining the particles with Ca²⁺ ions at or proximate a target site of a subject, e.g., to crosslink and form a gel matrix or other crosslinked material at the target site or to be subsequently administrated to the target site. The particles may be in the form of a suspension or slurry, e.g., an aqueous suspension or slurry, when combined with the with calcium ions.

The chitosan particles of the compositions herein may be have a particle size to facilitate administration to an internal site, e.g., via a catheter or other suitable medical device for endoscopic and/or urological delivery. Particle size may be characterized by morphological analysis, e.g., by static automated imaging. For example, a Malvern Morphologi 4 instrument may be used to analyze various particle parameters, including circle equivalent (CE) diameter, length, width, perimeter, area, maximum distance, sphere equivalent (SE) volume, and aspect ratio. As used herein, and only for the purposes of this disclosure, the term “average particle diameter” refers to the CE diameter, which is defined as the diameter of a circle with the same area as the particle. According to some examples herein, the chitosan particles may have an average particle diameter ranging from about 100 nm to about 700 μm, such as about 250 nm to about 700 μm, about 400 nm to about 700 μm, about 650 nm to about 700 μm, about 100 nm to about 600 μm, about 100 nm to about 500 μm, about 100 nm to about 400 μm, about 100 nm to about 300 μm, about 100 nm to about 200 μm, or about 100 nm to about 100 μm. In some examples herein, the average particle diameter may be less than about 700 μm, less than about 650 μm, less than about 600 μm, less than about 550 μm, less than about 500 μm, less than about 450 μm, less than about 400 μm, less than about 350 μm, less than about 300 μm, less than about 250 μm, less than about 200 μm, or less than about 150 μm.

The suspension or slurry of particles may comprise water (e.g., deionized water) and optionally one or more agents or fillers to increase viscosity and/or help keep the particles suspended to facilitate delivery. For example, the slurry or suspension may further comprise polyethylene glycol, a chitosan solution, a surfactant, or combinations thereof.

According to some aspects of the present disclosure, the slurry or suspension may comprise from about 1 mg/mL to about 50 mg/mL of chitosan particles (particles comprising chitosan and TPP), such as from about 1 mg/mL to about 45 mg/mL, from about 1 mg/mL to about 40 mg/mL, from about 1 mg/mL to about 35 mg/mL, from about 1 mg/mL to about 30 mg/mL, from about 1 mg/mL to about 25 mg/mL, from about 1 mg/mL to about 20 mg/mL, from about 5 mg/mL to about 25 mg/mL, from about 5 mg/mL to about 15 mg/mL, from about 10 mg/mL to about 30 mg/mL, from about 15 mg/mL to about 45 mg/mL, from about 20 mg/mL to about 40 mg/mL, from about 25 mg/mL to about 30 mg/mL, from about 30 mg/mL to about 50 mg/mL, or from about 35 mg/mL to about 45 mg/mL.

The suspension or slurry of particles may be combined with an aqueous calcium salt solution as a source of calcium ions. For example, the salt may comprise calcium chloride. In some examples, the salt solution may have a concentration ranging from about 0.1 M to about 2 M, such as from about 0.5 M to about 1.5 M, e.g., a concentration of about 1 M. According to some aspects of the present disclosure, the salt solution may be supersaturated.

The relative amounts of chitosan suspension or slurry and salt solution may be determined based on the nature of the target site, e.g., the size of the target site, and desired characteristics of the crosslinked material. In at least one example, 10-20 mL of a 1 M calcium chloride solution is combined with 10-30 mL of an aqueous suspension or slurry that includes 25 mg/mL chitosan particles (particles comprising chitosan and TPP).

As mentioned above, the structure of the gel/crosslinked material may be broken down if desired. Formation of the gel may be reversible, such that the crosslinked structure of the material may be broken down by adding saline solution. For example, the saline solution may disrupt the crosslinked structure afforded by the calcium ions and phosphate groups, e.g., to revert to a chitosan suspension or slurry if desired. This may be beneficial for removal of the composition if the composition is formed and/or administered to the wrong location, for example, or if excess composition remains following a medical procedure.

### EXAMPLES

The following examples are intended to illustrate the present disclosure without, however, being limiting in nature. It is understood that the present disclosure encompasses additional aspects and embodiments consistent with the foregoing description and following examples.

### Example 1

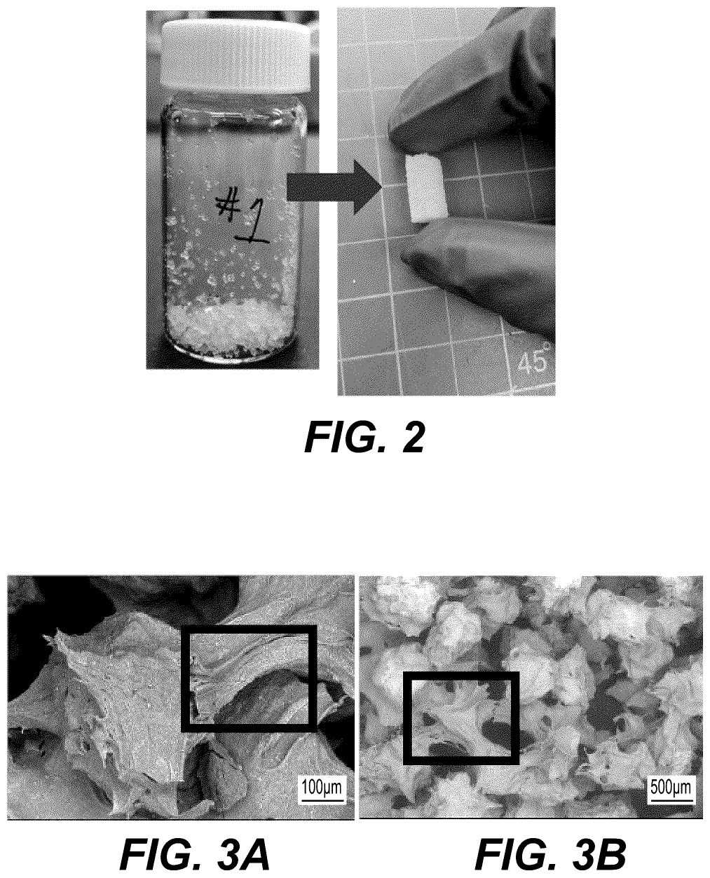

A crosslinked composition was prepared by combining particles comprising chitosan and TPP with a salt solution. The particles are shown in the left image of FIG. 2, wherein the particles were decanted from an aqueous solution. An amount of 0.2 g of the particles were soaked in 1 M calcium chloride solution, resulting in formation of a crosslinked matrix, shown in the right image of FIG. 2. Microstructure of the material was analyzed by scanning electron microscopy/energy dispersive spectroscopy (SEM/EDS) as shown in FIGS. 3A and 3B. The boxes shown in FIGS. 3A and 3B indicate relatively high CaCl₂) concentrations as measured by EDS, an indication of cross-bridging of calcium ions between particles. The size bar in FIG. 3A is 100 μm and the size bar in FIG. 3B is 500 μm. The crosslinked particles produced a spongy, conformable matrix expected to take shape of open space in the body.

Other aspects of the present disclosure will be apparent to those skilled in the art from consideration of the specification and practice of the examples disclosed herein. It is intended that the specification and examples be considered as exemplary only, with a true scope and spirit of the present disclosure being indicated by the following claims.

What is claimed is:

1. A method of treating a subject, the method comprising:
administering a composition to a target site of the subject, wherein administering the composition includes combining particles that comprise chitosan and tripolyphosphate with calcium ions to form a crosslinked material.

2. The method of claim 1, wherein the calcium ions are provided by a calcium salt solution.

3. The method of claim 1, wherein the calcium ions are calcium ions of a calcium chloride salt solution.

4. The method of claim 1, wherein the particles comprising chitosan and tripolyphosphate have an average particle diameter ranging from about 100 μm to about 700 μm.

5. The method of claim 1, wherein the target site is a fistula, and the composition at least partially or completely fills the fistula.

6. The method of claim 1, wherein the target site is soft tissue, and the composition separates tissue layers.

7. The method of claim 1, wherein the target site is between a pancreas and an abdominal cavity or gallbladder of the subject.

8. The method of claim 1, wherein the composition is administered via a catheter.

9. The method of claim 1, wherein administering the composition includes combining the particles with the calcium ions at the target site at the same time.

10. The method of claim 1, further comprising removing at least a portion of the composition from the target site by adding saline solution to the composition.

11. A method of preparing a composition, the method comprising combining particles that comprise chitosan and tripolyphosphate with a calcium salt solution to form the composition, wherein the composition comprises chitosan crosslinked with tripolyphosphate and calcium.

12. The method of claim 11, wherein the composition is prepared by combining an aqueous suspension of the particles with the calcium salt solution.

13. The method of claim 12, wherein the aqueous suspension further comprises polyethylene glycol, a surfactant, or both.

14. The method of claim 11, wherein the calcium salt solution comprises calcium chloride.

15. The method of claim 11, wherein the calcium salt solution is supersaturated.

16. The method of claim 11, wherein the particles comprising chitosan and tripolyphosphate have an average particle diameter ranging from about 100 μm to about 700 μm.

17. A method of treating a subject, the method comprising:
administering a composition to a target site of the subject by combining an aqueous suspension comprising particles that comprise chitosan and tripolyphosphate with a calcium salt solution at the target site to form a crosslinked material.

18. The method of claim 17, wherein the aqueous suspension further comprises polyethylene glycol, a surfactant, or both.

19. The method of claim 17, where the target site is a fistula or soft tissue of the urinary tract or gastrointestinal tract.

20. The method of claim 17, wherein the composition is administered via a catheter.
